(19) United States
(12) Patent Application Publication
(10) Pub. No.: US 2002/0099059 A1
(43) Pub. Date: Jul. 25, 2002

(54) Combination therapy for the treatment of migraine

(76) Inventor: Joel Saper, Ann Arbor, MI (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 09/934,276

(22) Filed: Aug. 21, 2001

Related U.S. Application Data
(60) Provisional application No. 60/227,350, filed on Aug. 23, 2000.

(51) Int. Cl. A61K 31/522

(52) U.S. Cl. 514/263.31

(57) ABSTRACT

A method of treating migraine and compositions useful therein are disclosed. The compositions comprise a selective 5-hydroxytriptamine receptor agonist and acetaminophen, non-steroidal anti-inflammatory agents and/or caffeine.

### CROSS REFERENCE TO RELATED APPLICATIONS

This non-provisional application claims priority from provisional application U.S. Ser. No. 60/227,350 filed Aug. 23, 2000.

### FIELD OF THE INVENTION

The present invention relates to compositions and methods for alleviating the symptoms and pain associated with acute migraine attack. More particularly, the invention relates to the use of acetaminophen (APAP), a nonsteroid antiinflammatory agent (NSAID) and/or caffeine (CAF), with a selective hydroxytriptamine₁ receptor subtype agonist or a selective 5-hydroxytriyptamine 1B/1D (5-HT 1B/1D) receptor agonist for alleviating the symptoms and pain associated with migraine.

### BACKGROUND OF THE INVENTION

An estimated 23-25 million Americans—about 18% of women and 6% of men—suffer from migraine pain and migraine-related symptoms. Attacks are common, with more than 50% of sufferers experiencing one or more episodes per month.

Migraine, a heterogeneous disorder, produces a wide spectrum of pain and associated disabilities, both within and among individual sufferers. The spectrum includes mild pain and no disability in approximately 5-15% of migraine attacks, moderate to severe pain and disability in approximately 60-70% of attacks, and incapacitating pain and total disability in the remaining approximately 25-35% of attacks.

Migraine is a disorder of the central nervous system characterized by unpredictable attacks of intense, pulsating head pain on one or both sides of the head. The majority of people who suffer from migraines report experiencing nausea, vomiting and, in some cases, sensitivity to light (photophobia) and to sound (phonophobia). Migraine attacks can last between two and twenty-four hours and in extreme cases, up to three days. Estimates are that 3 out of 4 migraine sufferers cannot function normally during an attack.

Although the symptom pattern varies among migraine sufferers, the severity of migraine pain justifies a need for vigorous therapy in the great majority of cases. Traditional therapy, such as ergotamine, although effective during a migraine attack, is known to become progressively less effective if its administration is delayed. Ergotamine is frequently combined with caffeine, a known analgesic adjuvant, to facilitate absorption of the ergot alkaloid. However, repeated dosing of ergotamine induces long lasting and cumulative vasoconstriction, thereby requiring careful instructions and management of individuals who take oral preparations for migraine attack. Because of cumulative toxicity of ergotamine and its derivatives, safer and/or more effective therapeutics for the treatment and prophylaxis of migraine have been sought.

A prescription of antimigraine medication which is an alternative to ergotamine and its derivatives is sumatriptan (or sumatriptan succinate). This material is a selective 5-hydroxytryptamine₁ receptor subtype agonist that is effective in a migraine attack. Other selective 5- hydroxytriptamine₁ receptor subtype agonists and other selective 5-hydroxytriptamine_{1B/1D} (5-HT_{1B/1D}) receptor agonists are employed in the treatment of migraine, however, all of these agents have the potential for significant side effects, and all suffer from one or more deficiencies.

U.S. Pat. No. 5,972,916 discloses oral compositions containing nonprescription amounts of acetaminophen, aspirin and caffeine (APAP/ASA/CAF) for alleviating the pain and symptoms of migraine. Also disclosed is the use of the APAP/ASA/CAF combination for aborting a migraine attack. U.S. Pat. No. 5,872,145 discloses methods of treating migraine by the co-timely administration of a 5-HT agonist and an NSAID or a nonNSAID.

There is need in the art for more effective anti-migraine drugs and treatments and particularly drugs and treatment offering greater pain relief, faster onset of activity and/or reduced side effects.

### SUMMARY OF THE INVENTION

It is an object of the present invention to provide compositions for treating migraine pain and at least one further symptom characteristic of a migraine attack, said symptom being selected from the group consisting of nausea, photophobia, phonophobia and functional disability, in a human in need thereof, comprising (i) a selective 5-HT₁ receptor subtype agonist or a selective 5-HT_{1B/1D} receptor agonist and (ii) APAP, NSAID and/or caffeine, in amounts effective to provide (i) more rapid onset of pain relief, (ii) greater pain relief (iii) longer-acting pain relief and/or (iv) pain relief to a greater population of users, than is obtained through use of said 5-HT agonists alone.

Another object of the present invention are methods for treating migraine pain and at least one further symptom characteristic of a migraine attack, said symptom being selected from the group consisting of nausea, photophobia, phonophobia and functional disability, in a human in need thereof, comprising the administration of a composition comprising (i) a selective 5-HT₁ receptor subtype agonist or a selective 5-HT_{1B/1D} receptor agonist and (ii) APAP, NSAID and/or caffeine, in amounts effective to provide (i) more rapid onset of pain relief, (ii) greater pain relief (iii) longer-acting pain relief and/or (iv) pain relief to a greater population of users, than is obtained through use of said 5-HT agonists alone.

Yet another object of the present invention are methods for treating migraine pain and at least one further symptom characteristic of a migraine attack, said symptom being selected from the group consisting of nausea, photophobia, phonophobia and functional disability, in a human in need thereof, comprising the co-administration of (i) a selective 5-HT₁ receptor subtype agonist or a selective 5-HT_{1B/1D} receptor agonist and (ii) APAP, NSAID and/or caffeine, in amounts effective to provide (i) more rapid onset of pain relief, (ii) greater pain relief (iii) longer-acting pain relief and/or (iv) pain relief to a greater population of users, than is obtained through use of said 5-HT agonists alone.

Another object of the present invention is to provide compositions and methods as described above for providing abortive relief of migraine during the prodrome or aura phases which precede the onset of migraine-associated symptoms and progression to an acute migraine.

Yet another object of the present invention is to provide compositions and methods as described above wherein said NSAID is ASA, ibuprofen, ketoprofen or naproxen.

Further objects and advantages afforded by the present invention will be apparent from the detailed description hereinbelow.

### DETAILED DESCRIPTION OF THE INVENTION

Selective 5-hydroxytriptamine₁ receptor subtype agonists are exemplified by sumatriptan and naratriptan. Sumatriptan is available as the succinate, under the trademark Imitrex®. Naratriptan is available as the hydrochloride salt, under the trademark Amerge®.

Examples of selective 5-HT_{1B/1D} receptor agonists are zolmitriptan and rizatriptan. Zolmitriptan is available under the trademark Zomig®. Rizatriptan is available as the benzoate, under the trademark Maxalt®.

Of the above-mentioned agonists sumatriptan is preferred. Sumatriptan is chemically identified as 3-(2-(dimethylamino)ethyl)-N-methyl-1-H -indole-5-ylmethyl sulfonamide succinate. It is the subject of Glaxo U.S. Pat. No. 4,816,470. Clinical evaluation of sumatriptan in migraine appears in Lancet 1, 309 (1988). Evaluation in acute headache appears in Lancet 1, 322 (1988). A further disclosure relative to the activity of sumatriptan is Headache 30 (Suppl. 2), 554-60 (1990). When sumatriptan succinate is employed as the agonist in the composition of the present invention, it is present in an amount of from 25 mg. to a 100 mg. of sumatriptan (base). Preferably it is present in an amount of from 25 to 50 mg. per dose. The size of the dosage unit may however be such the resultant tablet or capsule would be too large and would be difficult to swallow. When this occurs, the composition can be adjusted such that the dose of sumatriptan is provided by administering two tablets or two capsules rather than one tablet or capsule. This is a simple adjustment well known to those skilled in the art.

When naratriptan is employed as the agonist it is generally employed as the hydrochloride salt. Naratriptan is chemically designated as N-methyl-2-(3-(1 -methylpiperidin-4-yl)-1 H-indol-5-yl)ethane sulfonamide. Naratriptan is indicated for the acute treatment of migraine attacks with or without aura in adults. Naratriptan hydrochloride is presently marketed by Glaxo Wellcome under the trademark Amerge®. Amerge tablets are indicated as not intended for the prophylactic therapy of migraine or for use in the management of hemiphegic or basilar migraine. When naratriptan hydrochloride is employed as the agonist in the composition of the present invention, it is generally present in amount of from 1 to 2.5 mg. of naratriptan (base) as the hydrochloride.

Zolmitriptan, a 5-HT_{1B/1D} receptor agonist, is chemically designated as 4(s)-[3-(2-(dimethylamino0ethyl)-1H-indole-5-ylmethyl]oxazolidin-2one. Zolmitriptan is indicated for the acute treatment of migraine with or without aura in adults. When Zolmitriptan is employed as the agonist in the compositions of the present invention, it is present in an amount of from 1 to 5 mg. with 2.5 to 5 mg. being preferred. Zolmitriptan is available as Zomig tablets from AstraZeneca.

Rizatriptan benzoate is available from Merck and Company under the trademark Maxalt®. Rizatriptan benzoate is described chemically as 3-(2-dimethylamino)ethyl)-5-(1,2,4-triazol-1-ylmethyl)indole benzoate. Rizatriptan benzoate is indicated for the acute treatment of migraine attacks with our without aura in adults. When rizatriptan benzoate is employed as the agonist in the compositions of the present invention, it is present in an amount of from 5 to 10 mg. (corresponding to 7.265 mg. or 14.53 mg. of the benzoate salt, respectively).

Another agonist that can be employed in the composition of the present invention is eletriptan, which after approval is obtained will be marketed by Pfizer under the trademark, Relpax. Eletriptan is a selective serotonin (5-HT_{1B/1D}) agonist that reproduces the action of a neuro transmitter in the brain called seratonin or 5HT which is believed to constrict swollen blood vessels in the membranes covering the brain and interrupt the transmission of pain. Serotonin (5-HT_{1B/1D}) agonists activate sites in the swollen blood vessels—called serotonin 1B and 1D receptors—causing the vessels to constrict and reducing both the swelling and pain. Electriptan is described chemically as 5-(2-(benzenesulfonyl)ethyl)-3-(1-methylpyrrolidin-2-(R)-ylmethyl)-1H-indole. When eletriptan is employed as the agonist in the compositions of the present invention, it is present in an amount of from between 20 to 80 mg.

Another 5-HT_{1B/1D} agonist that can be employed in the compositions of the present invention is frovatriptan, a product of Vanguard Medica. Frovatriptan corresponds to the trademark product, Miguard®. The product is licensed to Elan in the United States. When frovatriptan is employed in the compositions of the present invention it is present in a dose of 2.5 to 40 mg.

Still another 5-HT_{1B/1D} agonist that can be employed in the composition of the present invention is almotriptan. This product of Almirall-Prosdespharma is licensed to Pharmacia and Upjohn. When almotriptan is employed in the compositions of the instant invention, it is present in a dose of 12.5 to 50 mg., preferably 12.5 to 25 mg.

The amount of the composition administered should be such as to be effective to reduce or eliminate the migraine pain and one or more of the symptoms characteristic of migraine. More preferably, the amount of the composition that is administered should be such as to be effective to significantly reduce the time prior to onset of relief of migraine pain as compared with the administration of the 5-HT agonist alone or the combination of APAP/NSAID/CAF alone. Alternatively, each of said ingredients of the invention may be provided in sub-therapeutic (subminimal effective dose) amounts or therapeutic (minimal effective dose) amounts.

The agonist component of the composition will be present in an amount which when combined with the APAP/ASA/CAF component results in a faster onset of migraine pain relief or that affords relief of migraine and at least one of its symptoms coupled with a lowered risk of side effects of the agonist component.

Particular amounts of said ingredients to achieve the various aspects of the invention comprise from about 200 mg. to about 600 mg., preferably about 400 mg. to about 550 mg.; and more preferably about 500 mg. of acetaminophen; about 300 mg. to about 600 mg.; preferably about 400 mg. to about 550 mg., and more preferably 500 mg. of aspirin and about 65 to 250, preferably about 65 to 200 mg., and more preferably, about 130 mg. of caffeine. A unit dose may be provided as needed to achieve said particular migraine-treating amounts.

Pharmaceutical preparations containing the composition of the invention and conventional pharmaceutical carriers may be employed in suitable unit dosage forms, such as solids or liquids. Solid form preparations include, for example, tablets, pills, caplets, capsules, powders, dispersible granules, cachets, and suppositories. Preferred are tablets, pills and capsules. Liquid form preparations include, for example, isotonic solutions, suspensions, or elixirs for oral administration or liquid solutions, suspensions and emulsions for parenteral use. The unit dosage form can be a package preparation, the package containing discreet quantities of preparation, for example, packeted tablets, capsules, and powders in vials or ampules. The unit dosage form can also be capsule, cachet, or tablet itself, or it can be the appropriate number of any of these in package form.

Tablets may contain the active ingredient in admixture with nontoxic, pharmaceutically acceptable excipients which are suitable for the manufacture of tablets. These excipients may be, for example, inert diluents, for example, calcium carbonate, sodium carbonate, lactose, calcium phosphate or sodium phosphate; granulating or disintegrating agents, for example, maize starch or alginic acid; binding agents, for example, starch, gelatin, or acacia; and lubricating agents, for example, magnesium stearate or stearic acid. The tablets may be uncoated or they may be coated by known techniques to delay disintegration and absorption in the gastrointestinal tract, and thereby provide a sustained action for a longer period of time.

In addition, tablets may contain the active preparation as a powder or granules, for example, a lyophilized powder or granules optionally mixed with binders, lubricants, inert diluents, or surface-active or dispersing agents, may be formed by compression or by milling in inert liquid diluent. Such tablets may be optionally scored and/or coated. Capsules and cachets may contain the active compounds alone or in admixture with one or more accessory ingredients. Capsules may also contain the active ingredients in aqueous or oleaginous solution, suspension, or emulsion, optionally in association with accessory ingredients.

Formulations for oral use may also be presented as hard gelatin capsules wherein the active components are mixed with an inert solid diluent, for example, calcium carbonate calcium phosphate, or kaolin, or as soft gelatin capsules, wherein the active ingredients are mixed with an oil medium, for example, arachis oil, liquid paraffin, or olive oil.

Additional formulations suitable for other modes of administration, such as suppositories, may include binders and carrier, for example, polyalkylene glycols or triglycerides.

Aqueous suspensions contain the active ingredients in admixture with excipients suitable for the manufacture of aqueous suspension. Nonlimiting example of such excipients include suspending agents, for example, sodium carboxymethyl cellulose, methylcellulose, hydroxypropylmethylcellulose, sodium alginate, polyvinylpyrrolidone, gum tragacanth, and gum acacia. Dispersing or wetting agents may be a naturally-occurring phosphatide, for example, lecithin, or condensation products of an alkylene oxide with fatty acids, for example, polyoxyethylene stearate, or condensation products of ethylene oxide with long chain aliphatic alcohols, for example, heptadecaethyleneoxycetamol, or condensation products of ethylene oxide with partial esters derived from fatty acids and a hexitol, for example, polyoxyethylene sorbitol monooleate, or condensation products of ethylene oxide with partial esters derived from fatty acids and hexitol anhydrides, for example, polyoxyethylene sorbitan monooleate. The aqueous suspensions may also contain one or more preservatives, for example, ethyl or n-propyl-phydroxy benzoate, one or more coloring agents, one or more flavoring agents, and one or more sweetening agents, such as sucrose, saccharin, or sodium or calcium cyclamate.

Several modes and routes of administration may be used to administer the composition in accordance with the present invention. For example, the composition in appropriate dosage form may be made available for oral, sublingual, nasal, rectal, intravenous, intramuscular, parenteral, rectal suppository or inhaler use. Oral administration is preferred.

The following Example illustrates a Tablet formulation in accordance with the present invention. Although the preferred agonist, sumatriptan, is illustrated and the formulation contains the preferred mixture of APAP/ASA/CAF, it should be appreciated that other agents falling within the scope of the present invention could be employed.

### Example of Tablet Formulation

| | | |
|---|---|---|
| Aspirin | 250.0 | mg. |
| Caffeine | 65.0 | mg. |
| Acetaminophen | 250.0 | mg. |
| Microcrystalline cellulose | 100.0 | mg. |
| Sumatriptan* succinate | 35.0 | mg. |
| Hydroxypropyl cellulose | 5.0 | mg. |
| Stearic Acid | 2.5 | mg. |
| | 707.5 | mg./Tab. |

*equivalent to 25 mg. of the free base

Tablets are prepared on a suitable Tableting machine. Preferably, the tablets are then film coated with a Dri-Klear film coating suspension.

What is claimed is:

1. A composition for treating migraine pain and at least one further symptom characteristic of a migraine attack, said symptom being selected from the group consisting of nausea, photophobia, phonophobia and functional disability, comprising (i) a selective 5-HT₁ receptor subtype agonist or a selective 5-HT_{1B/1D} receptor agonist and (ii) APAP, NSAID or caffeine, in amounts effective to provide (i) more rapid onset of pain relief, (ii) greater pain relief, (iii) longer-acting pain relief or (iv) pain relief to a greater population of users, than is obtained through use of said 5-HT agonists alone.

2. A composition for treating migraine pain and at least one further symptom characteristic of a migraine attack, said symptom being selected from the group consisting of nausea, photophobia, phonophobia and functional disability, comprising (i) a selective 5-HT₁ receptor subtype agonist or a selective 5-HT_{1B/1D} receptor agonist and (ii) APAP and NSAID, in amounts effective to provide (i) more rapid onset of pain relief, (ii) greater pain relief, (iii) longer-acting pain relief or (iv) pain relief to a greater population of users, than is obtained through use of said 5-HT agonists alone.

3. A composition for treating migraine pain and at least one further symptom characteristic of a migraine attack, said symptom being selected from the group consisting of nausea, photophobia, phonophobia and functional disability, comprising (i) a selective 5-HT₁ receptor subtype agonist or a selective 5-HT_{1B/1D} receptor agonist and (ii) APAP and caffeine, in amounts effective to provide (i) more rapid onset of pain relief, (ii) greater pain relief, (iii) longer-acting pain relief or (iv) pain relief to a greater population of users, than is obtained through use of said 5-HT agonists alone.

4. A composition for treating migraine pain and at least one further symptom characteristic of a migraine attack, said symptom being selected from the group consisting of nausea, photophobia, phonophobia and functional disability, comprising (i) a selective 5-HT₁ receptor subtype agonist or a selective 5-HT_{1B/1D} receptor agonist and (ii) NSAID and caffeine, in amounts effective to provide (i) more rapid onset of pain relief, (ii) greater pain relief, (iii) longer-acting pain relief or (iv) pain relief to a greater population of users, than is obtained through use of said 5-HT agonists alone.

5. A composition for treating migraine pain and at least one further symptom characteristic of a migraine attack, said symptom being selected from the group consisting of nausea, photophobia, phonophobia and functional disability, comprising (i) a selective 5-HT₁ receptor subtype agonist or a selective 5-HT_{1B/1D} receptor agonist and (ii) caffeine in amounts effective to provide (i) more rapid onset of pain relief, (ii) greater pain relief, (iii) longer-acting pain relief or (iv) pain relief to a greater population of users, than is obtained through use of said 5-HT agonists alone.

6. A composition for treating migraine pain and at least one further symptom characteristic of a migraine attack, said symptom being selected from the group consisting of nausea, photophobia, phonophobia and functional disability, comprising (i) a selective 5-HT₁ receptor subtype agonist or a selective 5-HT_{1B/1D} receptor agonist and (ii) APAP, NSAID and caffeine, in amounts effective to provide (i) more rapid onset of pain relief, (ii) greater pain relief, (iii) longer-acting pain relief or (iv) pain relief to a greater population of users, than is obtained through use of said 5-HT agonists alone.

7. The composition according to claim 6 in amounts effective to provide more rapid onset of pain relief than is obtained through use of said 5-HT agonists alone.

8. The composition according to claim 6 in amounts effective to provide greater pain relief than is obtained through use of said 5-HT agonists alone.

9. The composition according to claim 6 in amounts effective to provide pain relief to a greater population of users than is obtained through use of said 5-HT agonists alone.

10. The composition according to claim 6, wherein the 5-HT agonist is selected from the group consisting of sumatriptan succinate, naratriptan hydrochloride, zolmitriptan, rizatriptan benzoate, eletriptan, frovatriptan and almotriptan.

11. The composition according to claim 6, wherein the 5-HT agonist is sumatriptan succinate.

12. The composition according to claim 6, wherein the NSAID is aspirin.

13. The composition according to claim 6, wherein the NSAID is ibuprofen.

14. The composition according to claim 6, wherein a unit dose of the composition contains about 200 to about 300 mg acetaminophen, about 200 to about 300 mg aspirin and about 55 to about 100 mg. caffeine.

15. A method for treating migraine pain and at least one further symptom characteristic of a migraine attack, said symptom being selected from the group consisting of nausea, photophobia, phonophobia and functional disability, in a human in need thereof, comprising the administration of a composition according to claim 6.

16. A method for treating migraine pain and at least one further symptom characteristic of a migraine attack, said symptom being selected from the group consisting of nausea, photophobia, phonophobia and functional disability, in a human in need thereof, comprising the coadministration of (i) a selective 5-HT₁ receptor subtype agonist or a selective 5-HT_{1B/1D} receptor agonist and (ii) APAP, NSAID and caffeine, in amounts effective to provide (i) more rapid onset of pain relief, (ii) greater pain relief, (iii) longer-acting pain relief or (iv) pain relief to a greater population of users, than is obtained through use of said 5-HT agonists alone.
